(19) United States
(12) Patent Application Publication
(10) Pub. No.: US 2022/0304609 A1
(43) Pub. Date: Sep. 29, 2022

(54) PRE-OPERATIVE ELECTRODE DISPENSING NEURMONITORING GUN SYSTEMS AND METHODS

(71) Applicant: Creative Neural Solutions LLC, Glastonbury, CT (US)

(72) Inventors: Joseph Haddad, GLASTONBURY, CT (US); Payam Andalib, Storrs-Mansfield, CT (US); Benjamin Arnold, San Diego, CA (US); Brian Bowman, Carlsbad, CA (US); Henry Warder, San Francisco, CA (US); Ian Fried, San Diego, CA (US)

(21) Appl. No.: 17/700,510

(22) Filed: Mar. 22, 2022

Related U.S. Application Data
(60) Provisional application No. 63/165,329, filed on Mar. 24, 2021.

(51) Int. Cl. A61B 5/257; A61B 5/273

(52) U.S. Cl. CPC A61B 5/257; A61B 5/273; A61B 2560/0443; A61B 2560/06; A61B 2560/0431

(57) ABSTRACT

Exemplary pre-operative electrode-dispensing neuromonitoring gun systems and methods involve the administration of a neuromonitoring electrode to a patient. A system can include a dispensing device and a cartridge. The dispensing device can have a case, a trigger, and a pusher. The cartridge can have a plurality of electrodes. In some cases, an electrode can include two subdermal leads, a posterior connection port or plug, and a casing to house the connection port before placement and to cover the leads upon removal.

### CROSS-REFERENCES TO RELATED APPLICATIONS

[open]

### BACKGROUND OF THE INVENTION

Embodiments of the present invention relate generally to neuromonitoring systems and methods, and in particular encompass devices that enable an operator to both place and prepare neuromonitoring electrodes on a patient.

### BRIEF SUMMARY OF THE INVENTION

Pre-operative electrode-dispensing neuromonitoring gun systems and methods are disclosed here. Exemplary gun devices include two major parts: the neuromonitoring electrodes placed on the patient, and the instrument used to hold and dispense the electrodes.

In some cases, the electrodes include two subdermal leads designed to pierce the skin to pick up electrical signals. The electrodes have wing-like casings that internally house an electrical plug for an intermediary wire to attach upon electrode placement. The wing-like casing has peel-away, adhesive stickers on the exterior surfaces of the central casing. A string connects the peel-away covers on both casings, allowing the user to peel the covers and flap down the wings easily with one hand. The adhesive stickers then form a strong bond to the skin to prevent electrode movement. During electrode removal, the wing-like casings can continue to be turned, so the exterior sides now come together over the leads once they are removed from the patient. The exterior surface has casing channels that help cover the leads. This helps prevent the chance of a needle stick. The leads are connected to the electrical plug via a wired connection that rests in the electrode housing. A hinge between the casing and the housing allows the electrode casing's wing-like movement. The electrodes are comprised of conductive wiring to pass electrical signals, and non-conductive plastics comprising the body.

In one aspect, embodiments of the present invention encompass pre-operative electrode-dispensing neuromonitoring gun systems and related devices and cartridges, and methods for their use and manufacture. An exemplary pre-operative electrode-dispensing neuromonitoring gun system can include a dispensing device and a cartridge. The dispensing device can include a case, a trigger, and a pusher. The cartridge can include a plurality of electrodes, such as neuromonitoring electrodes. In some cases, the trigger has an internal/output (as proximity may change with use) portion, the pusher has a proximal portion, and the SAA portion of the trigger is coupled with the proximal portion of the pusher. In some cases, the dispensing device further comprises a cartridge slider that engages the cartridge. In some cases, the dispensing device includes a cartridge slider, a cartridge stop, and a compression spring disposed between the cartridge slider and the cartridge stop.

In some cases, at least one of the plurality of electrodes includes a housing, a first wing-like casing coupled with the housing via a first hinge, and a second wing-like casing coupled with the housing via a second hinge. In some cases, at least one of the plurality of electrodes includes a first lead and a second lead. In some cases, at least one of the plurality of electrodes includes a housing, a first wing-like casing coupled with the housing via a first hinge, a second wing-like casing coupled with the housing via a second hinge, a first lead coupled with the housing, and a second lead coupled with the housing. According to some embodiments, at least one of the plurality of electrodes includes a plug. In some embodiments, at least one of the plurality of electrodes includes a female plug. In some cases, at least one of the plurality of electrodes includes a male plug. According to some embodiments, at least one of the plurality of electrodes includes an electrode plug and an electrode housing, and the electrode plug and the electrode housing are provided together as a single solid body.

In another aspect, embodiments of the present invention encompass systems, devices, and methods for administering a neuromonitoring electrode to a patient. Exemplary methods may include actuating a trigger of a dispensing device of a pre-operative electrode-dispensing neuromonitoring gun system, so as to dispense the neuromonitoring electrode from the dispensing device to the patient. The neuromonitoring electrode can include a lead, and the lead pierces the skin of the patient. In some cases, the neuromonitoring electrode includes a housing, a first wing-like casing coupled with the housing via a first hinge, and a second wing-like casing coupled with the housing via a second hinge. In some cases, the neuromonitoring electrode includes a first lead and a second lead. In some cases, the neuromonitoring electrode includes a housing, a first wing-like casing coupled with the housing via a first hinge, a second wing-like casing coupled with the housing via a second hinge, a first lead coupled with the housing, and a second lead coupled with the housing. In some cases, the neuromonitoring electrode includes a plug. In some cases, the neuromonitoring electrode includes a female plug. In some cases, the neuromonitoring electrode includes a male plug. In some cases, the neuromonitoring electrode includes an electrode plug and an electrode housing, and the electrode plug and the electrode housing are provided together as a single solid body. In some cases, methods may include securing the neuromonitoring electrode to the skin of the patient with an adhesive sticker.

These and other embodiments are described in further detail in the following description related to the appended drawing figures.

### BRIEF DESCRIPTION OF THE DRAWINGS

Specific embodiments of the disclosed device, delivery systems, or methods will now be described with reference to the drawings. Nothing in this detailed description is intended to imply that any particular component, feature, or step is essential to the invention.

FIG. 1 depicts aspects of an electrode-dispensing neuromonitoring gun device, in accordance with some embodiments.

FIG. 2 illustrates aspects of an electrode-dispensing neuromonitoring gun system, in accordance with some embodiments.

FIG. 3 illustrates aspects of an electrode-dispensing neuromonitoring gun system, in accordance with some embodiments.

FIG. 4 illustrates aspects of an electrode-dispensing neuromonitoring gun system, in accordance with some embodiments.

FIG. 5 illustrates aspects of an electrode-dispensing neuromonitoring gun system, in accordance with some embodiments.

FIG. 6 illustrates aspects of an electrode-dispensing neuromonitoring gun device, in accordance with some embodiments.

FIG. 7 illustrates aspects of a neuromonitoring electrode, in accordance with some embodiments.

FIG. 8 illustrates aspects of a neuromonitoring electrode, in accordance with some embodiments.

FIG. 9 illustrates aspects of a neuromonitoring electrode, in accordance with some embodiments.

FIG. 10 illustrates aspects of a neuromonitoring electrode, in accordance with some embodiments.

FIG. 11 illustrates aspects of a neuromonitoring electrode, in accordance with some embodiments.

FIG. 12 illustrates aspects of a neuromonitoring electrode, in accordance with some embodiments.

FIG. 13 illustrates aspects of a neuromonitoring electrode, in accordance with some embodiments.

FIG. 14 depicts aspects of a neuromonitoring electrode, in accordance with some embodiments.

FIG. 15 depicts aspects of a neuromonitoring electrode, in accordance with some embodiments.

### DETAILED DESCRIPTION OF THE INVENTION

Embodiments of the present invention encompass systems and methods for placing electrodes on a patient, which can be used to assess neurological and electrical activity across a region of the patient.

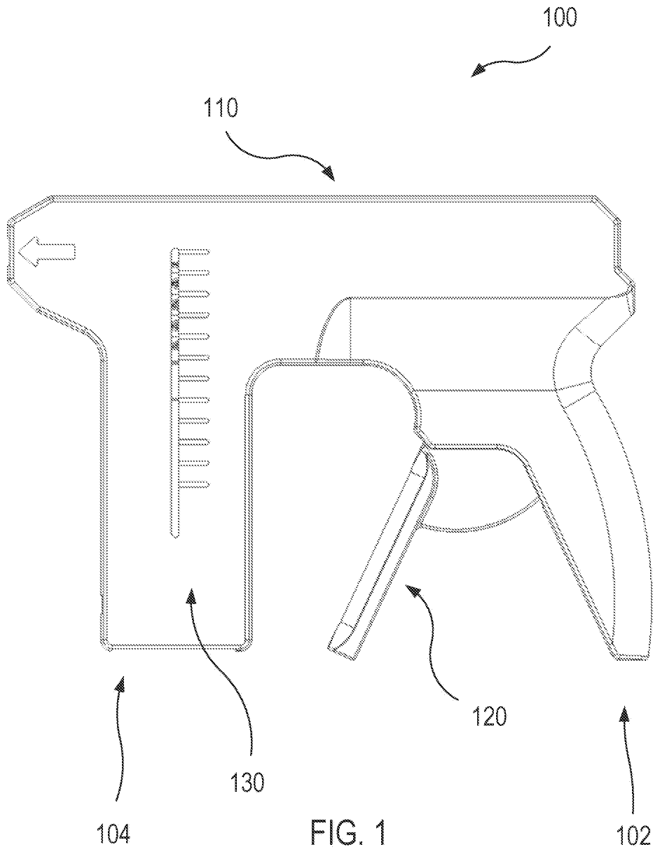

Turning now to the drawings. FIG. 1 depicts aspects of an electrode-dispensing neuromonitoring gun device 100, according to embodiments of the present invention. As shown here, the device 100 includes a handle/case 110, a trigger 120 (illustrated here in the “at rest” orientation), and a cartridge 130. The case 110 includes a proximal section 102 and a distal section 104. In some instances, device or instrument 100 can be defined as a box or box-like structure having approximate dimensions of 22 mm×120 mm×90 mm.

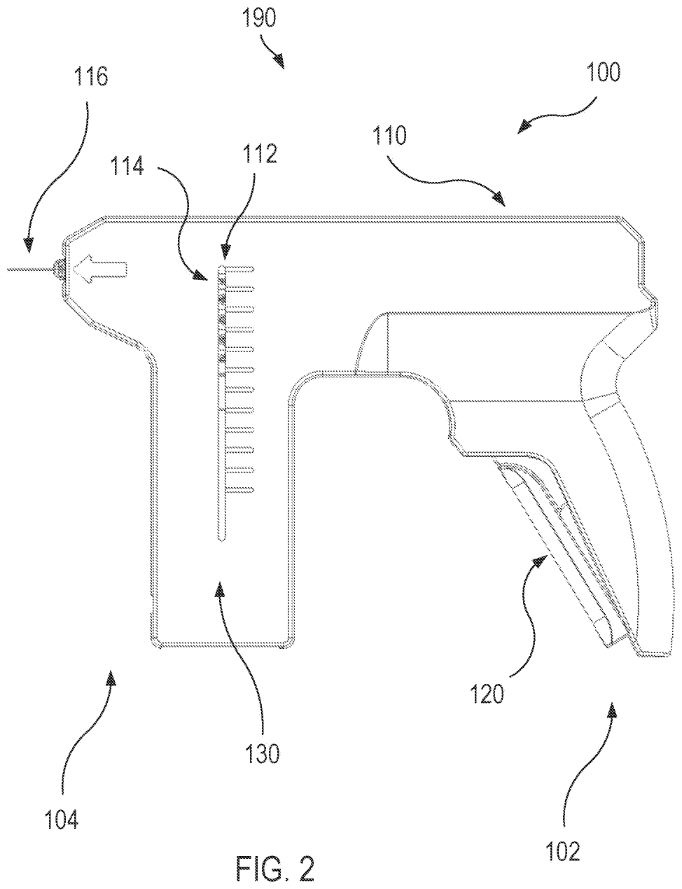

FIG. 2 depicts additional aspects of the electrode-dispensing neuromonitoring gun device 100, according to embodiments of the present invention. As shown here, the device 100 includes a handle/case 110, a trigger 120 (illustrated here in the “fired” orientation), and a cartridge 130. The case or casing 110 includes a window 112 through which an operator or user can view the number of remaining electrodes 114 that are held within the case 110. This illustration also shows a discharged electrode 16 exiting the distal section 104 of the case 110. In this sense, it can be seen that an embodiment for a system 190 can include two primary components, namely the neuromonitoring electrodes (e.g. 114, 116) that are placed on the patient, and the device or instrument 100 that is used to hold and dispense the electrodes.

Advantageously, embodiments of the present invention help to simplify the process of preparing a patient with neuromonitoring electrodes. Placement of electrodes can be performed by a user or operator using one hand, and preparation can be done with the other hand. Hence, what would otherwise be a slow and cumbersome process using existing techniques, can now be performed elegantly by a single person.

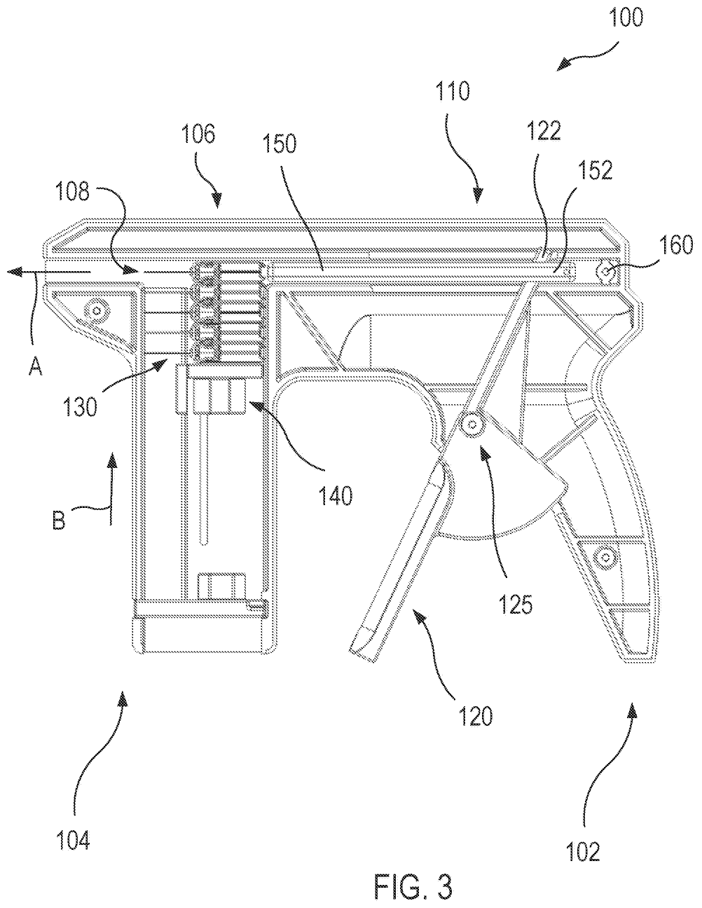

FIG. 3 depicts a cross-section view of the electrode-dispensing neuromonitoring gun device 100, according to embodiments of the present invention. As shown here, the device 100 includes a handle/case 110, a trigger 120 (illustrated here in the “at rest” orientation) in operative association with the case 110 via a fulcrum 125 about which trigger 120 pivots relative to case 110, and a cartridge 130. The case 110 includes a proximal section 102 and a distal section 104. In this image, a chambered electrode 108 is disposed in the case 110. In operation, a pusher 150 of the device 100 can be advanced from a proximal location toward the distal section 102 (e.g. upon activation or firing of the trigger 120) and the pusher 150 can contact the chambered electrode 108 and push it out of the case 110 in a distal direction, as indicated by arrow A. A proximal portion 152 of the pusher can be attached with a fixation point 160 of the case 110, for example via a spring. According to some embodiments, such a spring (and other subcomponents such as pins) can include or be fabricated from any of a variety of ductile metals, including without limitation stainless steel. The device 100 also includes a cartridge slider 140 that can operate to push the cartridge 130 toward an upper portion 106 of the case 110 in the direction indicated by arrow B. In some instances, the electrode pusher 150 can be biased in a rearward orientation and a proximal portion 122 of the trigger 120 (which is attached with proximal portion 152 of pusher 150) can also be biased in a rearward direction (e.g. when the trigger is at rest or unactuated) by an extension spring (not pictured) that is anchored between the fixation point 160 and the proximal portion 152 of the pusher 150. According to some embodiments, such a spring (and other subcomponents such as pins) can include or be fabricated from any of a variety of ductile metals, including without limitation stainless steel. In FIG. 3, there are five electrodes remaining in the cartridge 130, one of which is chambered. In some cases, the size of the cartridge 130 may vary. In some cases, the number of electrodes held by the device 100 or contained in the cartridge 130 can be variable.

As shown in the embodiment of FIG. 3, the cartridge 130 can extend perpendicularly off the bottom of the plane of insertion in a straight line. In other embodiments, the cartridge of the instrument could be positioned differently. For example, the cartridge could be extended in a different direction, and could additionally be curved. Relatedly, electrodes can also be provided with a tapered casing to allow for curved cartridge.

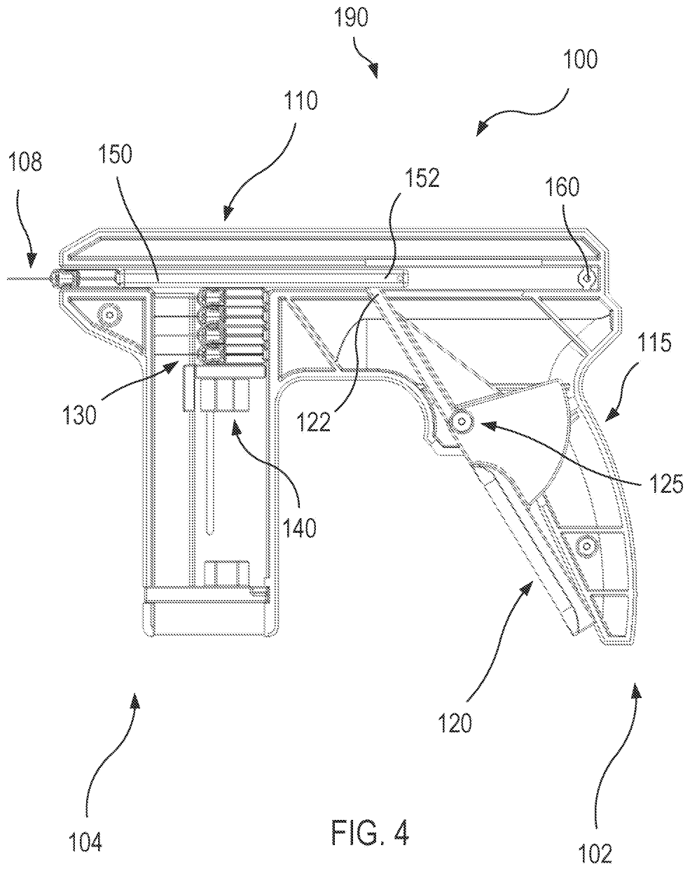

FIG. 4 depicts a cross-section view of the electrode-dispensing neuromonitoring gun device 100, according to embodiments of the present invention. As shown here, the device 100 includes a handle/case 110, a trigger 120 (illustrated here in the “fired” orientation) in operative association with the case 110 via a fulcrum 125 about which trigger 120 pivots relative to case 110, and a cartridge 130. The case 110 includes a proximal section 102 and a distal section 104. In this image, a chambered electrode 108 is exiting from the case 110. In operation, a pusher 150 of the device 100 can be advanced from a proximal location toward the distal section 102 (e.g. upon activation or firing of the trigger 120) and the pusher 150 can contact the chambered electrode 108 and push it out of the case 110 in a distal direction. A proximal portion 152 of the pusher can be attached with a fixation point 160 of the case 110, for example via a spring. The device 100 also includes a cartridge slider 140 that can operate to push the cartridge 130 toward an upper portion of the case 110. In some instances, the electrode pusher 150 can be biased in a rearward orientation and a proximal portion 122 of the trigger 120 (which is attached with proximal portion 152 of pusher 150) can also be biased in a rearward direction (e.g. when the trigger is at rest or unactuated) by an extension spring (not pictured) that is anchored between the fixation point 160 and the proximal portion 152 of the pusher 150. According to some embodiments, such a spring (and other subcomponents such as pins) can include or be fabricated from any of a variety of ductile metals, including without limitation stainless steel.

In order to appropriately assess all or a desired portion of the neurological and electrical activity across a region of the patient, many electrodes (e.g. 108) can be used. In some embodiments, the device or gun-like dispensing instrument 100 can be used to house and place twelve electrodes. Electrodes can be housed in a cartridge 130 from which they can be dispensed one at a time with use of the trigger 120 on the device or instrument 100. The device or instrument 100 can automatically prime the next electrode for placement (e.g. after a previous electrode is deployed). The device or instrument 100 can be made of injection molded plastics and simple springs.

According to some embodiments, the device or instrument 100 can include two injection molded casing halves that clam-shell together to form a case 110. The proximal section 102 of the instrument 100 can contain or include a handle 115 and can house or include a trigger 120 that can be fired or activated by the user to dispense electrodes. When fired, the trigger 120 can pivot about a fixed fulcrum 125 in the case or casing 110, causing the internal or proximal end or portion 122 of the trigger 120 to advance the pusher 150. The pusher 150 can extend part of the length of the trajectory plane of the device or instrument 100. When advanced, the pusher 150 can propel the chambered electrode 108 out of the open end of the device or instrument 100, and into the patient. There can be an extension spring that anchors the pusher 150 with a rear fixation point 160, and that can bias the pusher 150 and trigger 120 back to the resting configuration. According to some embodiments, such a spring (and other subcomponents such as pins) can include or be fabricated from any of a variety of ductile metals, including without limitation stainless steel. In some embodiments, the distal section 104 of the instrument 100 can be recognized as or include the cartridge 130, which can contain multiple electrodes and a mechanism to prime electrodes. As discussed elsewhere herein, an external window in a case or casing can enable or allow the user to see how many electrodes are remaining in the device or instrument. Advantageously, the system 190, which can be considered to include the device 100 and the cartridge 130 which contains or supports the electrodes, can provide a complete ensemble that allows one operator to both place and prepare the electrodes on the patient.

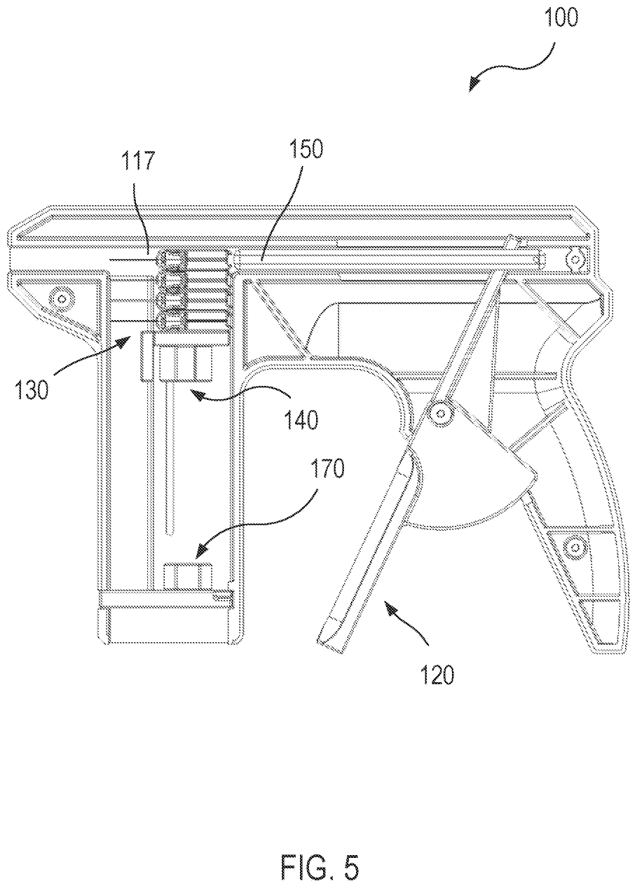

FIG. 5 depicts a cross-section view of the electrode-dispensing neuromonitoring gun device 100, according to embodiments of the present invention. As shown here, the device 100 includes a cartridge slider 140 and a cartridge stop 170. The slider 140 can be biased up along the cartridge 130 by a compression spring (not shown) that is anchored at or in contact with the stop 170. According to some embodiments, such a spring (and other subcomponents such as pins) can include or be fabricated from any of a variety of ductile metals, including without limitation stainless steel. In FIG. 5, there are four electrodes remaining in the cartridge 130, one of which is chambered. In operation, the cartridge slider 140 can be pushed up by a compression spring anchored at the cartridge stop 170 to bias the electrodes upwards and always ensure an electrode is chambered in front of or distal to the pusher 150. According to some embodiments, such a spring (and other subcomponents such as pins) can include or be fabricated from any of a variety of ductile metals, including without limitation stainless steel.

According to some embodiments, the device or instrument 100 includes a cartridge 130 or a similar housing compartment to hold multiple electrodes at once. The device 100 also can enable a method for placing a single electrode at a time, for example by activating a trigger 120 thereby dispensing electrodes from a chamber 117 of the case 110.

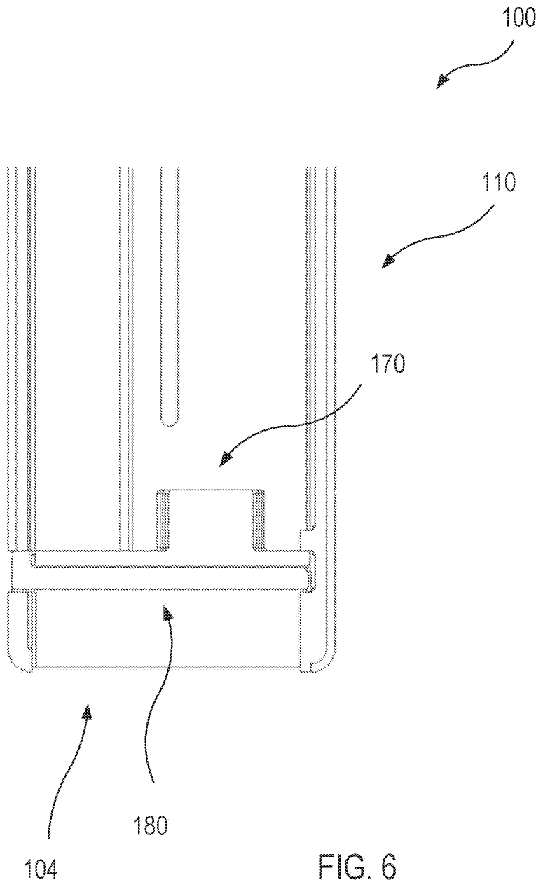

FIG. 6 depicts a partial cross-section view of the electrode-dispensing neuromonitoring gun device 100, according to embodiments of the present invention. As shown here, the device 100 includes a cartridge stop 170 and a cartridge cap 180, disposed in or engaged with the distal section 104 of the case 110. According to some embodiments, the cartridge stop 170 is assembled inferiorly, and then held in place by the cartridge cap 180, which is assembled from the front end or distal portion 104 of the instrument device 110. Hence, the cartridge cap 180 can operate to maintain the cartridge assembly.

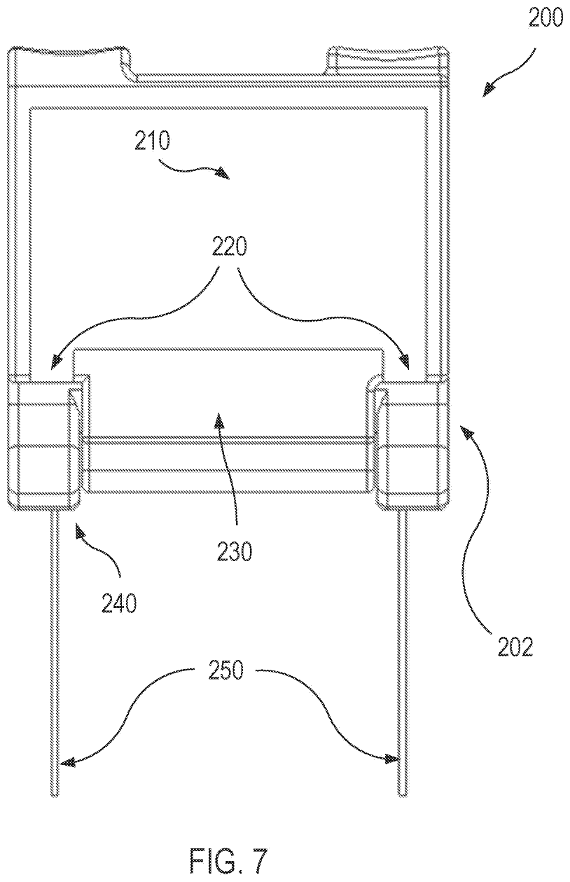

FIG. 7 depicts aspects of an electrode 200 for use with an electrode-dispensing neuromonitoring gun device, according to embodiments of the present invention. As shown here, the electrode 200 includes a central casing 210 for an adhesive sticker. The electrode 200 also includes casing channels 220, an electrode casing 230, an electrode housing 240, and subdermal leads 250. The casing channels 220 can operate to hold the leads 250 upon removal.

In exemplary embodiments, an electrode 200 includes two subdermal leads 250 designed to pierce the skin of a patient, and to pick up electrical signals. The leads 250, and other wiring components of an electrode or device, can include or be fabricated from any of a variety of conductive metals. Other components of the electrodes and device or instrument can include or be fabricated from any of a variety of plastics, including without limitation plastics that are able to be injection molded.

In some cases, an electrode 200 can be provided as a body 202 and leads 250, and the body can be defined as a box with dimensions of approximately 20 mm×18 mm×7 mm. The electrode leads 250 can extend from the body 202 a distance or length having a value of approximately 13 mm, making the complete box dimensions of the electrode 20 mm×31 mm×7 mm in some cases.

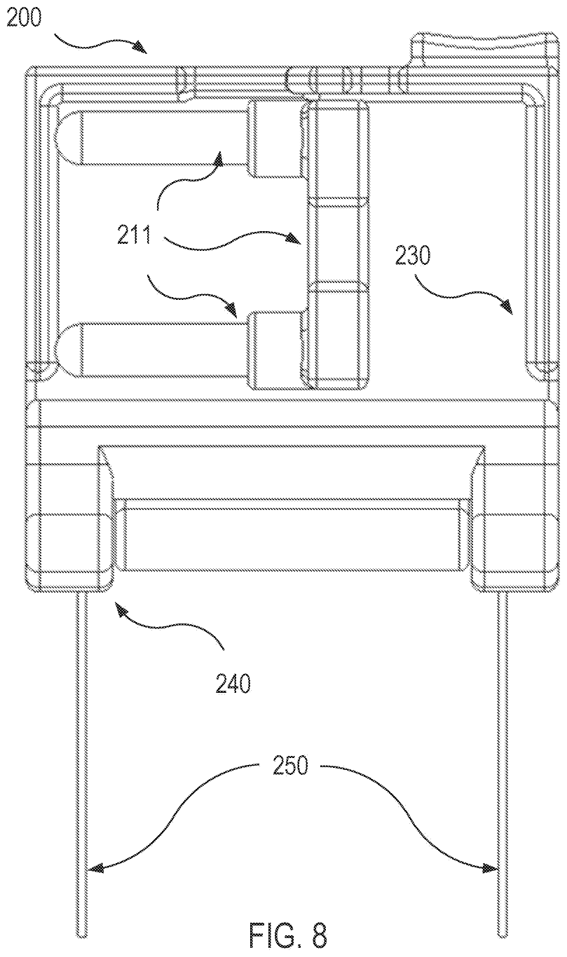

FIG. 8 depicts aspects of an electrode 200 for use with an electrode-dispensing neuromonitoring gun device, according to embodiments of the present invention. The electrode 200 includes an electrode plug 211 (male design), an electrode casing 230, and an electrode housing 240. According to some embodiments, the electrode plug 211 can be connected to the leads 250 with wires (not pictured) that enter the electrode housing 240 to meet the end of the leads 250.

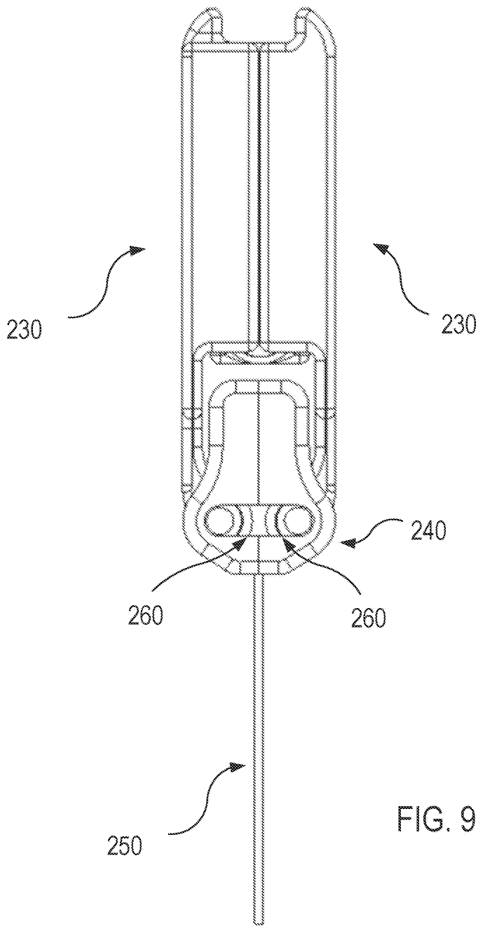

FIG. 9 provides a lateral view depicting aspects of an electrode 200 for use with an electrode-dispensing neuromonitoring gun device, according to embodiments of the present invention. As shown here, the electrode 200 includes hinges 260 disposed between casings 230 and a housing 240.

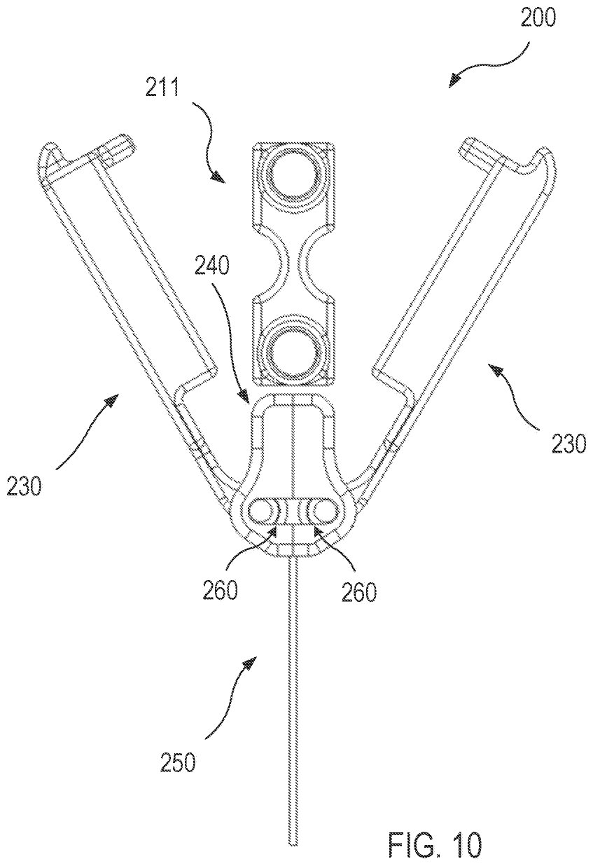

FIG. 10 provides a lateral view depicting aspects of an electrode 200 for use with an electrode-dispensing neuromonitoring gun device, according to embodiments of the present invention. As shown here, electrodes can have wing-like casings 230 that internally house an electrical plug 211 for an intermediary wire to attach upon electrode placement. The wing-like casing 230 has peel-away, adhesive stickers on the exterior surfaces of the central casing. A string connects the peel-away covers on both casings 230, allowing the user to peel the covers and flap down the wings easily with one hand. The adhesive stickers then form a strong bond to the skin of the patient to prevent electrode movement. The leads 250 are connected to the electrical via a wired connection that rests in the electrode housing 240. A hinge 260 between the casing 230 and the housing 240 allows the electrode casing's wing-like movement. In some cases, an electrode 200 can include conductive wiring to pass electrical signals, and non-conductive plastics comprising the body.

In exemplary embodiments, an electrode 200 can include two subdermal leads 250, a posterior connection port or plug 211, and a casing 230 to house the connection port 211 before placement and to cover the leads 250 upon removal. As further discussed below, an electrode 200 can be designed to naturally cover their sharp leads 250 as they are removed from the patient, making them safe from needle sticks.

In some cases, an electrode 200 can include or be operated in conjunction with a string that connects the two peel-away tabs on the adhesive sections. This string can be used to make removal of the tabs simple. A posterior connection port or plug 211 can be configured a wired connection between the plug 211 and the body of the electrode. This provides flexibility to the feature compared to a solid body connection, allowing easy manipulation of the plug 211 and prevents impingement if the electrode 200 is compressed between the patient and the bed. Optionally, the casing 230 for the electrode 200 can be provided as two hinged parts with 180° of rotation, and can simplify the needs between containing electrode components in shipping and placement and that of encasing the leads upon removal.

Advantageously, embodiments of the present invention encompass systems or electrodes that provide a single casing mechanism (e.g. casings 230) that can house or contain a connection port or plug 211 in distribution as well as cover sharps or leads upon removal, and can help keep the overall structure small by not needing additional components to perform these separate tasks. In some cases, connected peel-away covers on the electrode adhesives can make it easy for a single user to place, peel, and stick electrodes with one hand.

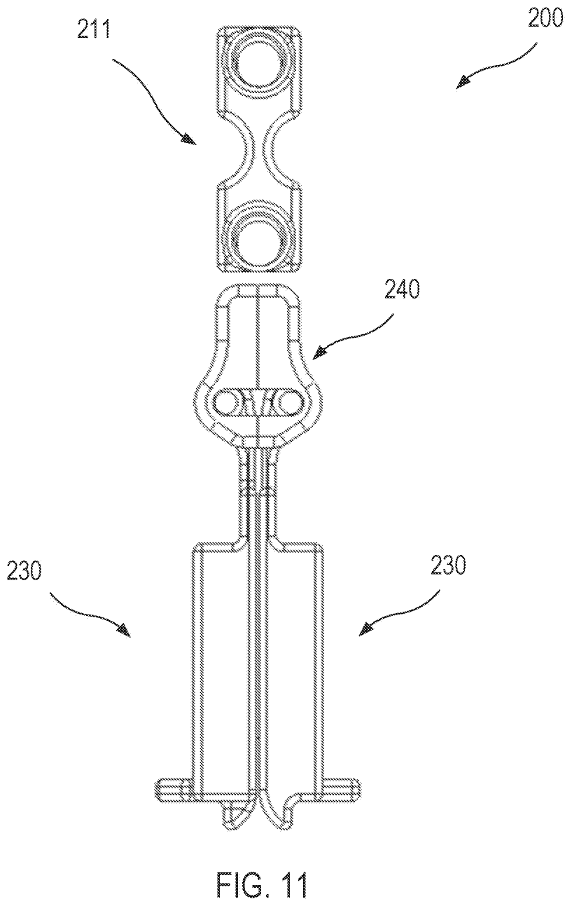

FIG. 11 provides a lateral view depicting aspects of an electrode 200 for use with an electrode-dispensing neuromonitoring gun device, according to embodiments of the present invention. During electrode removal, the wing-like casings 230 can continue to be turned, so the exterior sides now come together over the leads once they are removed from the patient. The exterior surface has casing channels that help cover the leads. This helps prevent the chance of a needle stick. Hence, it can be seen that advantageously, the two components (e.g. casings 230) that can be used to house the plug 211 on one side of the electrode body prior to use of the electrode 200 are the same components that can cover the leads after use.

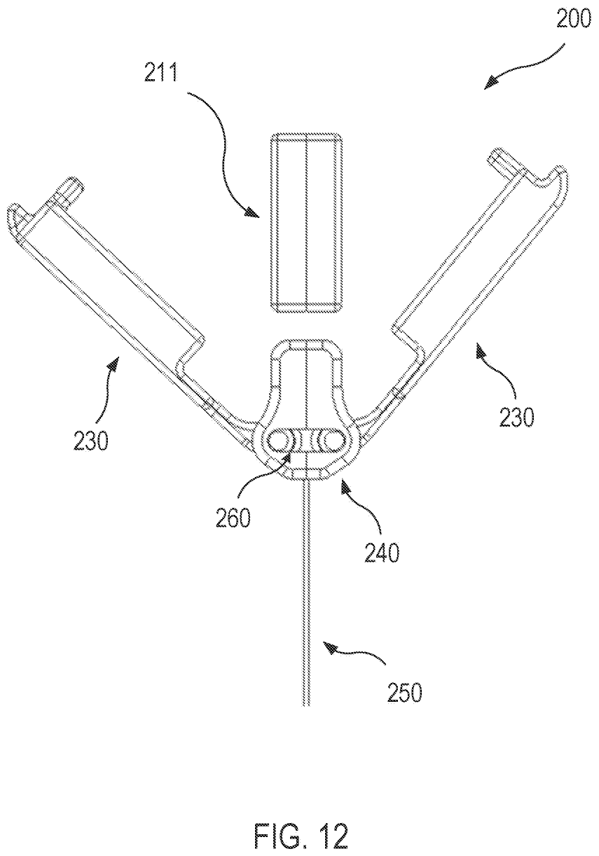

FIG. 12 provides a lateral view depicting aspects of an electrode 200 for use with an electrode-dispensing neuromonitoring gun device, according to embodiments of the present invention. As shown here, the electrode 200 can include an electrode plug 211 (female design).

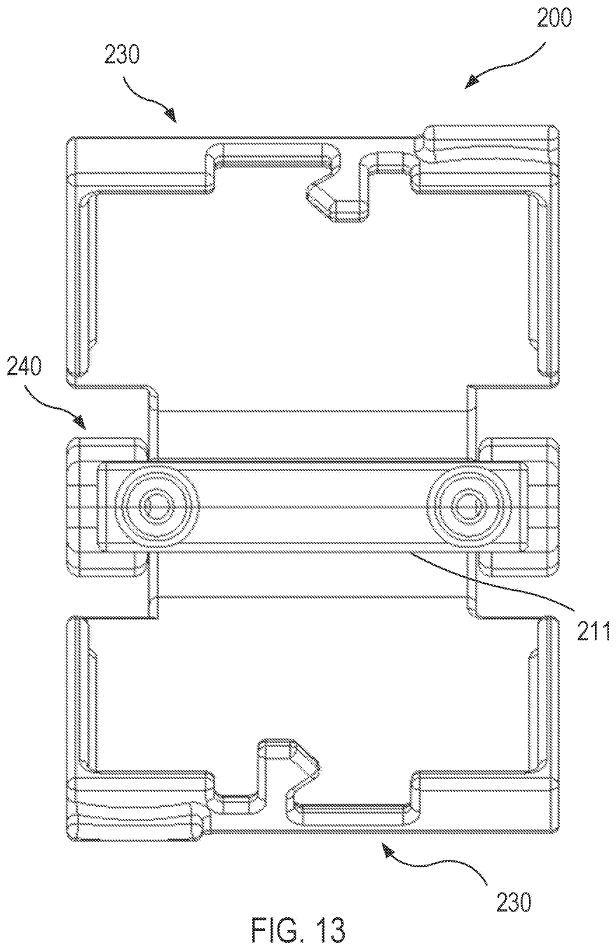

FIG. 13 provides a superior or top view depicting aspects of an electrode 200 for use with an electrode-dispensing neuromonitoring gun device, according to embodiments of the present invention.

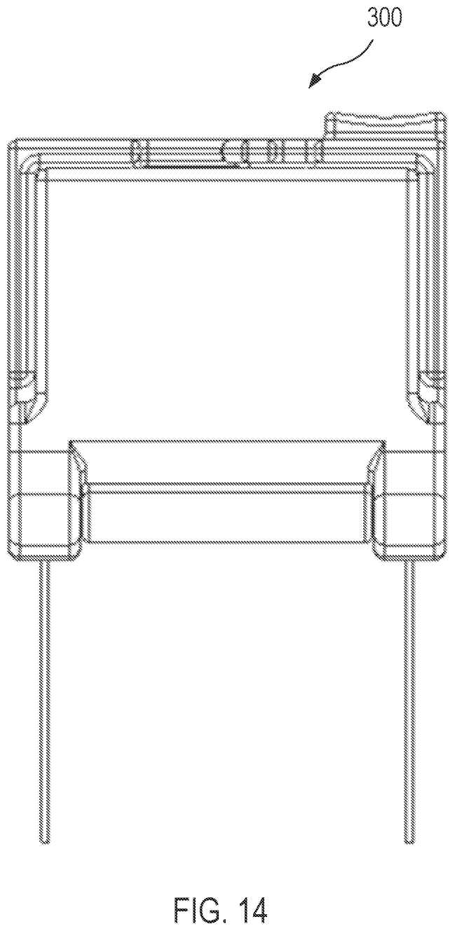

FIG. 14 provides a lateral view depicting aspects of an electrode 300 for use with an electrode-dispensing neuromonitoring gun device, according to embodiments of the present invention. In this embodiment, the electrode plug and electrode housing are provided together as a single solid body. In such an embodiment, a connection plug of the electrode 300 can have a solid connection to the electrode body. This may make the electrode 300 less convenient when moving the patient, but the solid connection would mean there is no need for a casing feature. This embodiment can provide an overall smaller electrode.

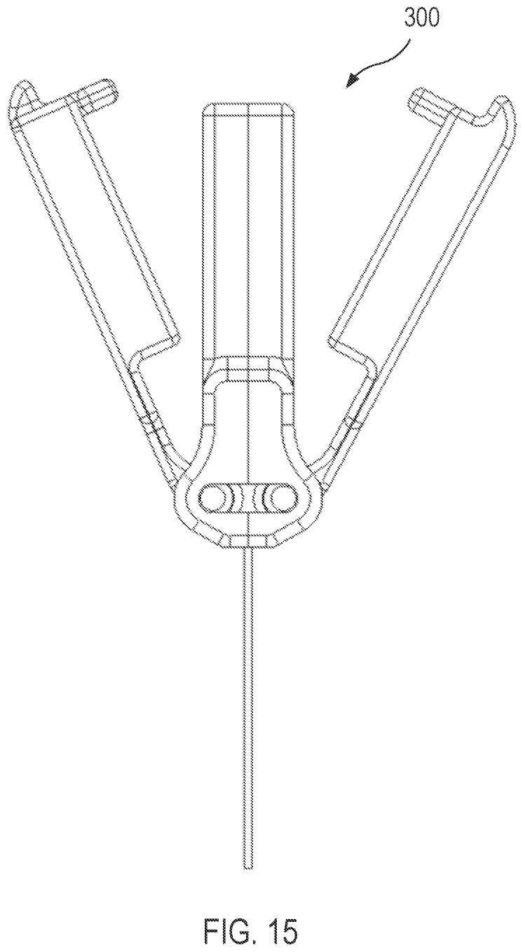

FIG. 15 provides a lateral view depicting aspects of the electrode 300 for use with an electrode-dispensing neuromonitoring gun device, according to embodiments of the present invention. In this embodiment, the electrode plug and electrode housing are provided together as a single solid body.

Although the preceding description contains significant detail in relation to certain preferred embodiments, it should not be construed as limiting the scope of the invention but rather as providing illustrations of the preferred embodiments.

Embodiments of the present invention encompass kits having pre-operative electrode-dispensing neuromonitoring gun systems as disclosed herein. In some embodiments, the kit includes one or more cartridges, gun devices, and/or systems, along with instructions for using the device(s) for example according to any of the methods disclosed herein.

All features of the described systems and devices are applicable to the described methods mutatis mutandis, and vice versa.

Although the foregoing invention has been described in some detail by way of illustration and example for purposes of clarity of understanding, one of skill in the art will appreciate that certain changes, modifications, alternate constructions, and/or equivalents may be practiced or employed as desired, and within the scope of the appended claims. In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

What is claimed is:

1. A pre-operative electrode-dispensing neuromonitoring gun system, comprising:
a dispensing device having a case, a trigger, and a pusher; and
a cartridge having a plurality of electrodes.

2. The pre-operative electrode-dispensing neuromonitoring gun system according to claim 1, wherein the trigger has a proximal portion, the pusher has a proximal portion, and the proximal portion of the trigger is coupled with the proximal portion of the pusher.

3. The pre-operative electrode-dispensing neuromonitoring gun system according to claim 1, wherein the dispensing device further comprises a cartridge slider that engages the cartridge.

4. The pre-operative electrode-dispensing neuromonitoring gun system according to claim 1, wherein the dispensing device further comprises a cartridge slider, a cartridge stop, and a compression spring disposed between the cartridge slider and the cartridge stop.

5. The pre-operative electrode-dispensing neuromonitoring gun system according to claim 1, wherein at least one of the plurality of electrodes comprises a housing, a first wing-like casing coupled with the housing via a first hinge, and a second wing-like casing coupled with the housing via a second hinge.

6. The pre-operative electrode-dispensing neuromonitoring gun system according to claim 1, wherein at least one of the plurality of electrodes comprises a first lead and a second lead.

7. The pre-operative electrode-dispensing neuromonitoring gun system according to claim 1, wherein at least one of the plurality of electrodes comprises a housing, a first wing-like casing coupled with the housing via a first hinge, a second wing-like casing coupled with the housing via a second hinge, a first lead coupled with the housing, and a second lead coupled with the housing.

8. The pre-operative electrode-dispensing neuromonitoring gun system according to claim 1, wherein at least one of the plurality of electrodes comprises a plug.

9. The pre-operative electrode-dispensing neuromonitoring gun system according to claim 1, wherein at least one of the plurality of electrodes comprises a female plug.

10. The pre-operative electrode-dispensing neuromonitoring gun system according to claim 1, wherein at least one of the plurality of electrodes comprises a male plug.

11. The pre-operative electrode-dispensing neuromonitoring gun system according to claim 1, wherein at least one of the plurality of electrodes comprises an electrode plug and an electrode housing, and wherein the electrode plug and the electrode housing are provided together as a single solid body.

12. A method of administering a neuromonitoring electrode to a patient, the method comprising:
actuating a trigger of a dispensing device of a pre-operative electrode-dispensing neuromonitoring gun system, so as to dispense the neuromonitoring electrode from the dispensing device to the patient, wherein the neuromonitoring electrode comprises a lead, and wherein the lead pierces the skin of the patient.

13. The method according to claim 12, wherein the neuromonitoring electrode comprises a housing, a first wing-like casing coupled with the housing via a first hinge, and a second wing-like casing coupled with the housing via a second hinge.

14. The method according to claim 12, wherein the neuromonitoring electrode comprises a first lead and a second lead.

15. The method according to claim 12, wherein the neuromonitoring electrode comprises a housing, a first wing-like casing coupled with the housing via a first hinge, a second wing-like casing coupled with the housing via a second hinge, a first lead coupled with the housing, and a second lead coupled with the housing.

16. The method according to claim 12, wherein the neuromonitoring electrode comprises a plug.

17. The method according to claim 12, wherein the neuromonitoring electrode comprises a female plug.

18. The method according to claim 12, wherein the neuromonitoring electrode comprises a male plug.

19. The method according to claim 12, wherein the neuromonitoring electrode comprises an electrode plug and an electrode housing, and wherein the electrode plug and the electrode housing are provided together as a single solid body.

20. The method according to claim 12, further comprising securing the neuromonitoring electrode to the skin of the patient with an adhesive sticker.
